(19) United States
(12) Patent Application Publication
(10) Pub. No.: US 2007/0043284 A1
(43) Pub. Date: Feb. 22, 2007

(54) Medical electrode and method of use

(76) Inventor: Robert Sanfilippo, Glendale, CA (US)

Correspondence Address: PATENT LAW & VENTURE GROUP, 2424 S.E. BRISTOL, SUITE 300, NEWPORT BEACH, CA, 92660, US

(21) Appl. No.: 11/205,894

(22) Filed: Aug. 16, 2005

(51) Int. Cl. A61B 5/04

(52) U.S. Cl. 600/391; 600/392

(57) ABSTRACT

A medical electrode provides enablement for simultaneously connecting a plurality of medical electrical monitoring or testing instruments at a selected single location on a patient's body. The electrode includes a conductive layer with tabs mounted on an adhesive insulating layer attached by adhesion to the patient's skin. Apertures in the layers are sized to accept a conductive snap-action terminal. The tab portions extend outwardly from the post for connecting with conventional electrical clips of auxiliary instruments. The tab portions and their clip attachments do not physically interfere with a connector's engagement to the central electrical terminal.

### BACKGROUND

1. Field of the Present Disclosure

This disclosure relates generally to medical devices and more particularly to a medical electrode with plural connection points for attachment of several monitoring devices simultaneously.

2. Description of Related Art

Medical electrodes are utilized in a number of applications for a variety of diagnostic and monitoring purposes. For instance, electrodes commonly are used to monitor physiological electric potentials to detect muscular activity of a person's heart. The cardiovascular activity of the heart is typically monitored by adhering or connecting electrodes to the skin of the patient at particular locations of interest on the body. The electrodes are then electrically coupled to electrical equipment such as an electrocardiograph (also referred to as EKG) apparatus that monitors the muscular activity of the heart. The resulting traces or output of the EKG provides a diagnostic tool for detecting heart disease and/or heart dysfunction of various etiology.

My prior patent, U.S. Pat. No. 5,626,135 describes the uses of medical electrodes and applications in the prior art quite thoroughly. U.S. Pat. No. 5,626,135 is hereby incorporated into the present document by reference.

### SUMMARY

A need exists for a medical electrode to which several electrical instruments can be simultaneously attached so as to monitor physiological electrical potentials at a specific location on a patient's skin surface. In accordance with one aspect of the present medical electrode an electrically conductive layer has a centrally positioned terminal and a plurality of tab portions peripheral thereto. An insulation layer is mounted to the skin of the patient and the electrically conductive layer is mounted on top of it. The conductive layer provides a conductive surface for electrically interconnecting a plurality of clip electrodes to the central terminal which is able to then make electrical contact with a primary monitoring equipment via a snap-on connector, and also to the skin of the patent via a conductive gel or similar substance between the central terminal and the patient's skin. In accordance with additional aspects of the present invention, a medical electrode is provided for simultaneous use with a primary electrical instrument and a plurality of auxiliary electrical instruments. The medical electrode includes an electrically conductive contact layer which supports an electrically conductive terminal. The terminal includes an engagement end, which is distal to the base. Plural electrically conductive tabs of the medical electrode are in electrical common with the terminal and the skin of the patient.

One object of the present apparatus is to provide benefits and improvements not known in prior art devices.

A further objective is to provide such an apparatus that is able to connect a plurality of monitoring devices to a single area of a patent's skin surface for electrical contact.

A still further objective is to provide such an apparatus that is able to provide electrical contact to a large number of electrical clips without physical interference with a central snap-on type terminal connector.

A still further objective is to provide such an apparatus that is easy to apply and remove, is inexpensive to manufacture and has a long shelf life.

### BRIEF DESCRIPTION OF THE DRAWINGS

These and other features of the invention will now be described with reference to the drawings of preferred embodiments which are intended to illustrate and not to limit the invention, and in which:

FIG. 1 is a perspective view of a medical electrode in accordance with a preferred embodiment of the present invention;

FIG. 2 a side cross-sectional view of the medical electrode shown in FIG. 1 taken along lines 2-2; and

FIG. 3 is a plan view of an alternate embodiment of the medical electrode.

### DETAILED DESCRIPTION

The above described drawing figures illustrate the described apparatus and its method of use in at least one of its preferred, best mode embodiments, which is further defined in detail in the following description. Those having ordinary skill in the art may be able to make alterations and modifications to what is described herein without departing from its spirit and scope. Therefore, it should be understood that what is illustrated is set forth only for the purposes of example and that it should not be taken as a limitation in the scope of the present apparatus and method of use.

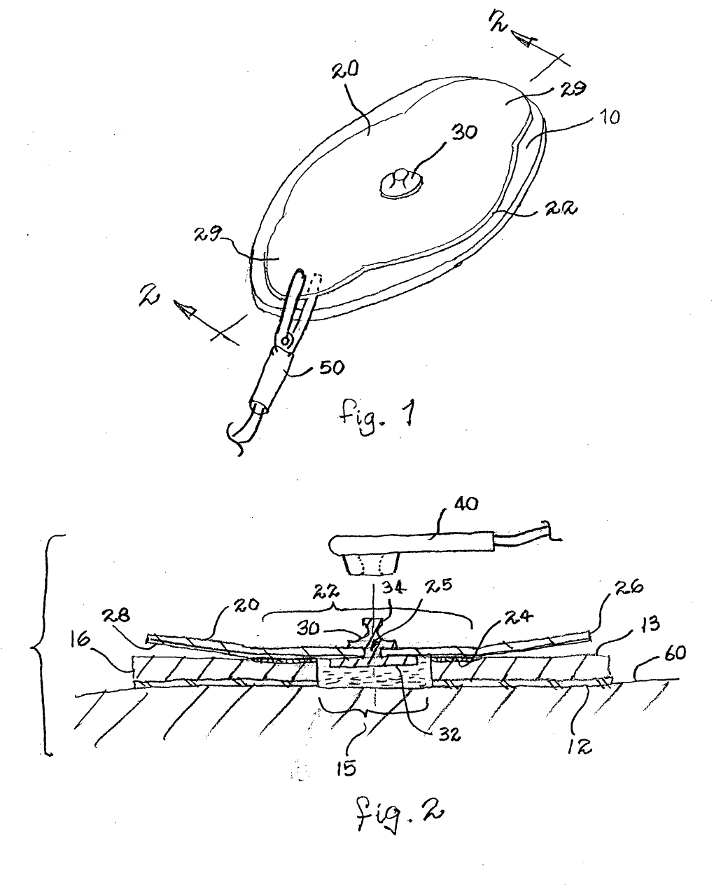

The apparatus described and claimed herein is a medical electrode for simultaneous use with a primary electrical monitoring instrument such as an EKG, and a plurality of auxiliary electrical instruments. The medical electrode, as shown in FIG. 1, comprises a first element which is a flexible, electrically insulating, adhesive layer 10 providing, as shown in FIG. 2, an adhesive material 12 on one side and an adhesive free surface 13 on an opposing side. Layer 10 has a medially, or centrally positioned first aperture 15, and a first encircling peripheral edge 16. A second element of the electrode is a flexible contact layer 20 which provides a medial portion 22 fixed to the adhesive layer 10, preferably by a bonding agent 24, in opposition, i.e., on the opposing side of the adhesive layer 10. The layer 20 has a second, at least partially encircling peripheral portion 26 which is not fixed to the adhesive layer 10. The contact layer 20 provides a conductive surface or layer 28 between the second encircling peripheral portion 26 and the medial portion 22. The conductive surface 28 preferably is coated onto or bonded to the entire surface of layer 20 which faces layer 10. The medial portion 22 of the contact layer 20 provides a second aperture 25 positioned coaxially with the first aperture 15 of the adhesive layer 10. The first encircling peripheral edge 16 is extensive of the second encircling peripheral edge 26 so that the conductive surface 28 is not able to touch any surface other than the top surface of the adhesive layer 10, thereby preventing an electrical short circuit.

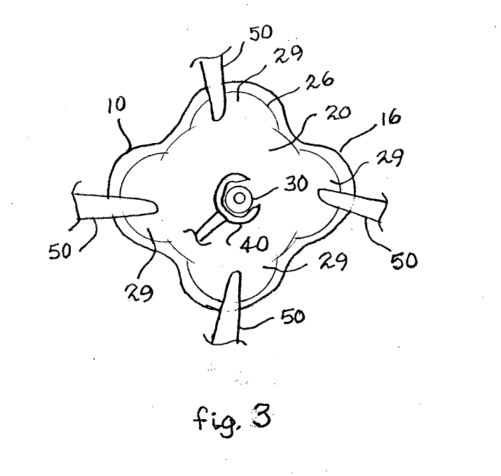

Preferably, the second encircling peripheral portion 26 of the contact layer 20 is formed into a plurality of contact tabs 29 as shown in FIGS. 2 and 3 which may be lifted away from the adhesive layer 10. Such contact tabs 29 may be spaced apart as shown in FIG. 2, or they may be positioned more closely to each other as shown in FIG. 3.

An electrically conductive terminal 30 is preferably engaged within the first aperture 15 of the adhesive layer 10 and is positioned within the second aperture 25 of the contact layer 20 as shown in FIG. 2. The terminal 30 is mounted to the contact layer 20 in a manner well known in the art, and provides an electrical contact surface 32 within the first aperture 15. Such snap-action type terminals are well known in the prior art and are described in the incorporated reference, U.S. Pat. No. 5,626,135. The terminal 30 is positioned and oriented such that a post portion 34 extends away from the contact layer 20, as shown in FIG. 2, so that it may be engaged with a female coupling 40 for making electrical contact with monitoring equipment while not interfering with clip type electrical contacts 50 engaged with the tabs 29 peripheral to the female coupling 40, as shown in FIGS. 1 and 3.

The materials of construction, type of construction and operating enablements of the present apparatus will be understood from the reference, U.S. Pat. No. 5,626,135. However, the present apparatus clearly improves on the embodiments taught in this reference by providing the adhesive layer 10 extensive of the contact layer 20 to prevent electrical shorts, by providing a larger aperture 15 in the adhesive layer 10 so as to improve the ability of the terminal 30 to make electrical contact with the skin surface 60, and by providing a plurality of tabs so that plural instruments may be joined in electrical parallel interconnection simultaneously.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. A medical electrode for simultaneous use with a primary electrical instrument and a plurality of auxiliary electrical instruments, the medical electrode comprising: a flexible, electrically insulating, adhesive layer providing an adhesive bottom surface, a centrally positioned first aperture, and a first encircling peripheral edge; and a flexible contact layer providing a medial portion fixed to a top surface of the adhesive layer, and a second encircling peripheral portion not fixed to the adhesive layer; the contact layer providing a conductive surface between the second encircling peripheral portion and the medial portion; the medial portion of the contact layer providing a second aperture coaxial with the first aperture of the adhesive layer; the first encircling peripheral edge extensive of the second peripheral edge.

2. The electrode of claim 1 wherein the peripheral portion of the contact layer is formed into a plurality of contact tabs not adhered to the adhesive layer.

3. The electrode of claim 2 wherein the contact tabs are spaced apart.

4. The electrode of claim 2 wherein the contact tabs are positioned in side-by-side adjacency.

5. The electrode of claim 1 further comprising an electrically conductive terminal engaged within the first aperture of the adhesive layer and the second aperture of the contact layer, the terminal extending away from the contact layer for engagement with a female coupling.
